(19) United States
(12) Patent Application Publication
(10) Pub. No.: US 2021/0117881 A1
(43) Pub. Date: Apr. 22, 2021

(54) NATURAL LANGUAGE WORKFLOW CONSTRUCTION

(71) Applicant: Bizagi Group Ltd, Sliema (MT)

(72) Inventors: Jesus Orlando Sanchez Merchan, Bogotá (CO); Gustavo Ignacio Gomez Gonzalez, Bogotá (CO); Marcel Josef Manser Sonderer, Bogotá (CO); Diego Enrique Parra Leal, Bogotá (CO); Anwar Saud Hussein Ibañez Okamell, Bogotá (CO); Jose Alcides Aranzazu Montoya, Bogotá (CO)

(21) Appl. No.: 16/076,156

(22) PCT Filed: Jan. 24, 2017
(86) PCT No.: PCT/EP2017/051362

§ 371 Date: Aug. 7, 2018

(30) Foreign Application Priority Data
Feb. 8, 2016 (GB) 1602260.0

(51) Int. Cl. G06Q 10/06; G06F 40/211; G06F 40/295; G16H 40/20; G06F 16/25

(52) U.S. Cl. CPC G06Q 10/0633; G06F 40/211; G06F 16/252; G16H 40/20; G06F 40/295

(57) ABSTRACT

A computer-implemented method for designing a workflow for use in workflow management software having access to a workflow database that includes one or more data objects, actions for operation on data objects, and a plurality of stakeholder objects each associated with (a) one or more of the data objects and (b) one or more of the actions, the method comprising: receiving at a user interface of a computer system a natural language input and an indication of a stakeholder represented in the workflow database by a stakeholder object; parsing the natural language input at a processor so as to identify a verb and a noun in the natural language input; verifying whether a data object corresponding to the noun exists in the workflow database; and if such an entity type does not exist in the workflow database: using a predefined data object, creating in the workflow database a new data object for the identified noun; using a predefined process definition, storing in the workflow database an action for the identified verb; and linking in the workflow database the new data object, the action for the identified verb, and the stakeholder object such that, when the workflow management software is in use by the stakeholder, the stakeholder is permitted to operate the action on an instance of the new data object associated with the stakeholder.

### BACKGROUND OF THE INVENTION

This invention relates to a method and computer system for designing a workflow for workflow management software.

Computer systems for workflow management are crucial to the efficient operation of businesses and organisations around the world. Many business use computer systems to define and organise processes which are to be followed by their personnel. Particularly in large organisations with workers spread over many sites or even countries, this can help to ensure that processes are consistently and efficiently performed throughout the business. For example, in service industries such as insurance or banking, the use of workflow management systems can ensure that a minimum quality of service is provided by the business to their customers. Workflow management may also be referred to as business process management (BPM).

A new class of workflow management software has recently become available which enables complex workflows to be enforced at a computer system without requiring every possible permutation of a business process to be predefined in the system. One approach is to define the stakeholders involved in a workflow, along with the actions those stakeholders may perform and the data over which those stakeholders have ownership and on which they can run their actions. The actions are not linked together so as to form predefined workflows; rather the workflows emerge from suitable definitions of the actions, data and rules governing when the actions and data are available to the stakeholders permitted to run them. This stakeholder-centric approach is embodied in BPM software available from Bizagi.

The new approach to workflow management provides substantial benefits over conventional systems for business process management but, like any workflow management system, still requires careful definition of the data set used by the software to enforce workflows.

### SUMMARY OF THE INVENTION

According to a first aspect of the present invention there is provided a computer-implemented method for designing a workflow for use in workflow management software having access to a workflow database that includes one or more data objects, actions for operation on data objects, and a plurality of stakeholder objects each associated with (a) one or more of the data objects and (b) one or more of the actions, the method comprising:
receiving at a user interface of a computer system a natural language input and an indication of a stakeholder represented in the workflow database by a stakeholder object;
parsing the natural language input at a processor so as to identify a verb and a noun in the natural language input;
verifying whether a data object corresponding to the noun exists in the workflow database; and
   if such an entity type does not exist in the workflow database:
using a predefined data object, creating in the workflow database a new data object for the identified noun;
using a predefined process definition, storing in the workflow database an action for the identified verb; and
linking in the workflow database the new data object, the action for the identified verb, and the stakeholder object such that, when the workflow management software is in use by the stakeholder, the stakeholder is permitted to operate the action on an instance of the new data object associated with the stakeholder.

Each stakeholder may be permitted to operate on a data object associated with the stakeholder only those actions which are linked to that data object and the stakeholder object representing the stakeholder.

The parsing the natural language input at a processor may be performed in dependence on one or more of: the position of the words in the natural language input; the presence of a connector word delineating a verb and noun; in which fields of a user interface the words of the natural language input are present.

The parsing the natural language input at a processor may comprise processing the natural language input using a natural language processing algorithm configured to identify a verb and a noun on which the verb syntactically operates.

The verifying whether a data object corresponding to the noun exists in the workflow database may comprise checking the data objects defined in the workflow database for a data object satisfying one or more of the following the conditions: being identified by the noun; tagged with the noun; having an identifier predefined as being equivalent to the noun.

The linking in the workflow database may comprise defining in the new data object that the stakeholder represented by the stakeholder object is permitted to operate the identified verb action on the new data object.

The receiving at a user interface may further comprise receiving an indication of a context condition and the linking in the workflow database comprises defining in the new data object that the stakeholder represented by the stakeholder object is permitted to operate the identified verb action on the new data object only when the context condition is satisfied.

The natural language input may be received at a user interface relating to a stakeholder, the receiving an indication of a stakeholder comprising identifying the stakeholder as the stakeholder to which the user interface relates.

The user interface relating to a stakeholder may be a design interface configured to allow a user to modify the stakeholder object representing the stakeholder.

The predefined data object may be a default data object, the new data object inheriting the properties of the default data object and being identified amongst the data objects of the workflow database by the noun.

The predefined process definition may be a default process definition, the action inheriting the properties of the default process definition and being identified amongst the actions of the workflow database by the verb.

The method may comprise, subsequent to the linking, prompting the user to edit attributes of one or more of the new data object and the action for the identified verb.

The process definitions may be defined at a process layer of the workflow database and the data objects are defined at a data layer of the workflow database.

According to a second aspect of the present invention there is provided a computer system for designing a workflow for use in workflow management software having access to a workflow database that includes one or more data objects, actions for operation on data objects, and a plurality of stakeholder objects each associated with (a) one or more of the data objects and (b) one or more of the actions, the system comprising:
a data store comprising the workflow database;
a user interface for receiving a natural language input and an indication of a stakeholder represented in the workflow database by a stakeholder object;
a processor configured to:
   parse the natural language input so as to identify a verb and a noun in the natural language input;
   verify whether a data object corresponding to the noun exists in the workflow database; and
if such an entity type does not exist in the workflow database:
   using a predefined data object, creating in the workflow database a new data object for the identified noun;
   using a predefined process definition, storing in the workflow database an action for the identified verb; and
   linking in the workflow database the new data object, the action for the identified verb, and the stakeholder object such that, when the workflow management software is in use by the stakeholder, the stakeholder is permitted to operate the action on an instance of the new data object associated with the stakeholder.

There may be provided computer program code for performing a method for designing a workflow for use in workflow management software. There may be provided a non-transitory computer readable storage medium having stored thereon computer readable instructions that, when executed at a processor, cause the processor to perform a method for designing a workflow for use in workflow management software.

### BRIEF DESCRIPTION OF THE DRAWINGS

The present invention will now be described by way of example with reference to the accompanying drawings. In the drawings:

FIG. 1 shows a computer system for designing a workflow according to the principles described herein.

FIG. 2 shows a workflow management system.

FIG. 3 illustrates an exemplary structure of the database of FIG. 2.

FIG. 4 illustrates a user interface of a terminal of FIG. 2.

FIG. 5 shows an example of a workflow performed at the system of FIG. 2.

FIG. 6 shows an example stakeholder object.

FIG. 7 shows an example data object.

FIG. 8 shows an example process definition.

FIG. 9 is a flowchart illustrating the operation of the workflow management system.

FIG. 10 is a flowchart illustrating a method of designing a workflow.

FIG. 11 shows an example user interface of a computer system for designing a workflow.

### DETAILED DESCRIPTION OF THE INVENTION

The following description is presented by way of example to enable a person skilled in the art to make and use the invention. The present invention is not limited to the embodiments described herein and various modifications to the disclosed embodiments will be apparent to those skilled in the art.

Although it is no longer necessary in stakeholder-centric workflow management systems to exhaustively define every possible permutation of a business process, when designing workflows for such a system it has been established that it can be difficult for non-technical users new to the system to appreciate how to appropriately define and configure each of the actions and data objects in order to enforce the desired sets of workflows in the system. It is desirable to ensure that non-technical users can design workflows for a workflow computer system, since the personnel in an organisation with the knowledge of business processes are often not technical and do not have a background in computer programming.

There is provided a method and computer system for designing a workflow for a workflow management system. The method and computer system are particularly suitable for designing workflows for workflow management systems which use the stakeholder-centric approach. The method and computer system address the above-identified difficulties which can be experienced by users new to designing workflows by providing a novel mechanism by which new actions and data objects can be generated at design time for a workflow management system.

Workflow Management System

By way of context and example, the stakeholder-centric approach used in workflow management systems will now be described. Such systems permit complex workflows to be enforced whilst minimising the storage and processing requirements of the computer system. Workflows need not be predefined at the system and instead emerge from the particular configuration of the computer system. Each workflow represents a sequence of actions which a group of one or more users of the system can collectively follow. As is described below, sequences of actions which are not permitted cannot be performed by users of the system because at any given step in a workflow, invalid actions are not presented to a user. In this manner the computer system can restrict users to performing valid workflows.

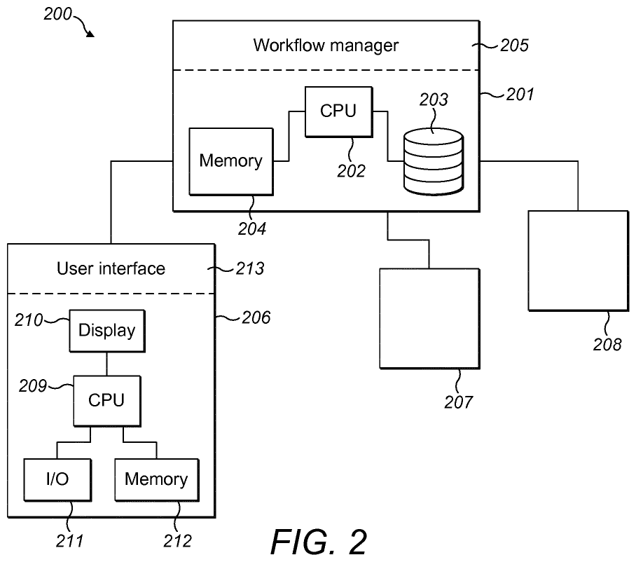

An example of a computer system for enforcing a workflow is shown in FIG. 2. In FIG. 2, computer system 200 comprises a computer server 201 having a processor 202, a memory 204 and a database 203. The database could be located at the server as shown in FIG. 2 or at a data store accessible to the server (e.g. over a network or the internet). The processor and memory support a workflow manager 205 which in this example is software arranged to operate on the data held at database 203 so as to allow users of computer terminals 206, 207 and 208 of the system to perform workflows.

There could be any number of terminals, but in this example there are three. The database could comprise one or more data stores distributed over the system, including locally at the server and/or at storage locations outside the server. The server could comprise one or more processing entities (e.g. processor cores, servers or blades on a blade server) with access to database 203 and supporting a workflow manager. The database may be a relational database, and associations between objects of the database may be represented by links between tables of the database.

Each of the computer terminals may be configured as shown for terminal 206 so as to include a processor 209, a display 210, an input/output unit 211 and a memory 212. The processor and memory support a user interface 213 with which a user of the terminal can interact with the computer terminal by means of input/output unit 211 (e.g. a keyboard, mouse, touchscreen, microphone etc.). Each terminal could be any kind of computing device, such as a desktop or laptop computer, a tablet or a smartphone. Each terminal could be a “thin” terminal which relies on the server to perform at least some of its processing, or a standalone computing device connected to server 201. It will be appreciated that many other configurations of computer server and terminal are possible.

The user interface 213 of a terminal presents the user with access to database 203. The user interface could provide a lightweight front-end (e.g. a web application providing the user interface 213) by means of which a user can interact with the workflow manager 205 which is configured to mediate access to the database 203 and substantially perform the processing required by a sequence of actions making up a workflow. In other examples, the user interface itself could include some or all of the workflow manager and hence perform some or all of the processing associated with a workflow. Many intermediate configurations are possible between these two exemplary extremes. The processors of the computer system (e.g. at the server and/or terminals) as configured by the workflow manager and/or user interface enforce workflows in accordance with the principles described herein.

One or more parts of database 203 could be located at the terminals as copies of part of the database which are accessible to the user interface at that respective terminal and/or by way of distributing at least some of the data of database 203 over the terminals of the system. The server could itself operate as a terminal by means of which a user can perform one or more steps of a workflow.

User interface 213 represents an example of a computer interface of the system 200 for providing users of the system access to data held at the database in the data objects. In other examples, the interface could be supported at the same processing entity as the workflow manager 205 and/or could be non-graphical, such as an application programming interface (API), web API, or similar. For instance, a workstation could comprise both a workflow manager and a user interface running at a processor, the workstation thus performing the functions of both server and terminal.

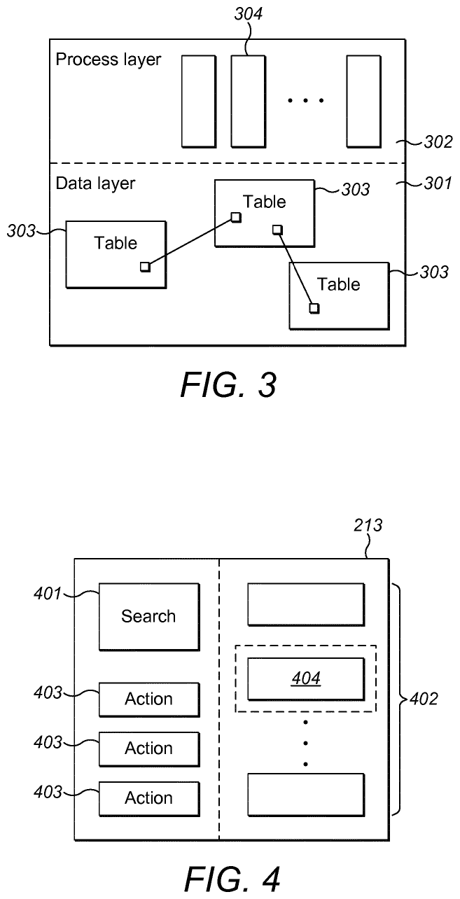

An example of the data held at database 203 is shown in FIG. 3. The database 203 may include a data layer 301 and a process layer 302. The data layer may include tables 303 defining a set of data objects and a set of stakeholder objects. The data objects and stakeholder objects may be linked so as to logically define for each stakeholder a set of data object instances associated with that stakeholder. Each stakeholder object (or an instance thereof) corresponds to a stakeholder role in the particular workflows which the computer system is configured to support. In some examples a stakeholder object may comprise multiple instances, each instance corresponding to a different stakeholder role—e.g. a stakeholder object may be provided for doctors in the hospital example below, with each particular doctor corresponding to a different stakeholder instance.

The tables may link to other data sources external to the database (e.g. data sources holding patient records, or contact details for the customers of a business). The process layer includes a set of process definitions 304 each of which specifies an action that can represent a step of a workflow. The process definitions may link to the tables in the data layer so as to reference the data and stakeholder objects, and vice versa. For example, the data objects may, by virtue of appropriate links from tables 303 to process definitions 304, identify actions permitted for operation on the data object and the stakeholders permitted to initiate each of those actions. The data and stakeholder objects, and the process definitions are described in more detail below. Other data structures able to define data and stakeholder objects and process definitions for use in the manner described herein may alternatively be used. The terms data layer and process layer may indicate logical groupings of data at a database and need not imply any corresponding structure of the database.

The workflow manager 205 is configured to operate in dependence on the data held at the database 203 so as to enforce workflows by constraining users of the computer terminals 206-208 to perform sequences of actions on data objects of the database in accordance with the permissions defined at the database. This may be achieved according to the following example.

A set of stakeholder objects are defined at the database 203, each defining a stakeholder in the workflows and the data objects which are associated with that stakeholder. Each stakeholder may represent a type of user of the system who is able to perform one or more actions at the system. For example, in a computer system configured to enable hospital staff to follows process in a hospital environment, the stakeholders may be the nurses, doctors and other hospital staff involved in performing actions at the system. Triage nurses might be one stakeholder, emergency room (ER) doctors a second stakeholder, and orthopaedic surgeons a third stakeholder. Each user of the system may belong to one or more stakeholders and there could be one or more users associated with each stakeholder.

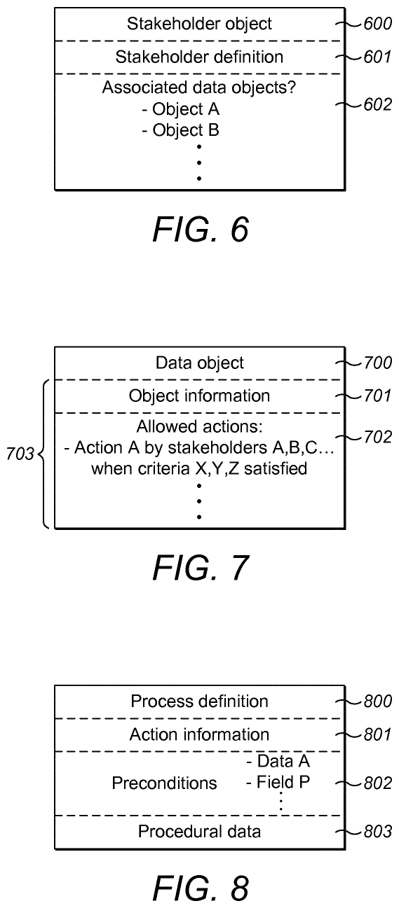

An example of the logical data content of a stakeholder object or definition 600 is shown in FIG. 6. The stakeholder object 600 includes a definition of the stakeholder 601, which might include information such as the name of that stakeholder and other characteristics. The stakeholder object may include data 602 specifying the data objects associated with the stakeholder (e.g. the orthopaedic surgeon mentioned above might be associated with data object instances representing surgical cases). In some examples, a stakeholder object 600 may comprise multiple stakeholder instances—for example, where a stakeholder represents a particular role (e.g. a doctor) and there are many individuals in such a role (e.g. many doctors in a hospital), each such individual may correspond to a particular stakeholder instance. Each instance may include a definition 601 and specification data 602 identifying the data object instances associated with that stakeholder. According to the particular implementation, a stakeholder may be represented by a stakeholder object or instances of a stakeholder object.

In other examples, information specifying the data objects may be stored in any other manner—for example, by linking the stakeholder object to its associated data objects, or storing such associations at specification data held at the database. A stakeholder object could include data defining one or more configurations of the user interface for the stakeholder—for example, the stakeholder object could define a configuration (e.g. appearance, arrangement of presented data, ability to browse data, and/or search data) of user interface 213 appropriate to the role performed by that stakeholder.

One or more data objects may be defined at the database 203. Each data object may represent a different type of entity—e.g., in the hospital example described below, one data object may represent X-Rays and another may represent patient appointments. Each data object may comprise multiple instances of the data object each of which may be associated with a different stakeholder (e.g. the X-Ray records of the patients in the system). Data at a data object instance may identify one or more actions which can be performed on that instance and/or this may be defined through appropriate linking of the data object instances to actions held at the process layer. A data object may collectively define the actions which may be performed on its instances, and may define stakeholders, types of stakeholders or groups of stakeholders who may perform those actions. Each data object instance may specify which stakeholders are permitted to run each of the actions which can be performed on the data object—e.g. through appropriate linking of the data object instance to stakeholder objects or instances held at the data layer. For example, in the workflow management system modelling workflows in a hospital, the data objects could be patient records, represent an investigation (e.g. an X-Ray, an MRI scan, a blood test), or be an appointment.

The database may comprise multiple types of data object. New instances of a data object (e.g. a new patient record) may inherit some or all of the properties of other data objects of the same type. For example, the workflow manager may be configured to associate a new instance of a data object with the same set of stakeholder objects and/or may set the permissions of a new instance of a data object such that the same set of actions can be run on the new instance as on the other data objects of the same type. In some implementations, a definition may be stored for each type of data object defining the associations and permissions of its instances; the data object instances of that type of data object may be considered to include that definition.

An example of the logical data content of a data object 700 is shown in FIG. 7. The data object includes information 701 about the object—for example, for a patient record data object this information might include the name, address, health system number and the medical records of the patient. Specification data 702 specifies the actions which can be performed on the object, which stakeholders can perform each of those actions, and optionally, in what context those actions can be performed. Object information 701 and the specification data 702 may be provided for each instance 703 of a data object.

Data objects and stakeholder objects as described herein may be any logical collections of data and may or may not be objects of a relational database. Data objects and stakeholder objects may be held in any manner at the computer system, including as one or more files or entries within files, or at a database.

A user of a computer terminal 206-208 is represented at database 203 as one or more corresponding stakeholders. A user terminal may be configured to authenticate a user (e.g. by requiring credentials from the user) so as to identify a profile stored for the user at the terminal or server. The computer system may be configured to associate each user profile with one or more stakeholder objects (or instances) held at the database so as to allow the system to identify which stakeholders a user of a computer terminal represents, and hence the data and actions which may be presented to that user in accordance with the principles described herein. Each user profile may be stored in the database and linked to the one or more stakeholder objects representing the stakeholders associated with the user of that user profile.

A user is restricted by the workflow manager to accessing only those data object instances associated with the stakeholder represented by the user. A user may access data objects at a user terminal in any suitable manner, including by browsing their data object instances, searching within data objects for data, or selecting data presented to them. The interface through which a user accesses their data may be configured to permit access in more than one way. As has been described, the user interface 213 may be configured according to the particular requirements of the stakeholder associated with the user.

Actions are defined in the database 203 as process definitions, each action being for operation on one or more data objects. An example of the logical data content of a process definition 800 defining an action is shown in FIG. 8. The process definition may include information about the action 801 (e.g. its name and other parameters), and procedural data 803 which defines the action itself—e.g. instructions defining the processing to be performed by the computer system, such as modifying, creating or deleting a data object instance, presenting a form to a user and enforcing the entry of mandatory information etc. Such procedural data could take any form, including, for example, data for interpretation at a processor of the server or client into a web application for presentation at the user interface, database operations, or any kind of program code suitable for execution at a processor of the server or user terminal.

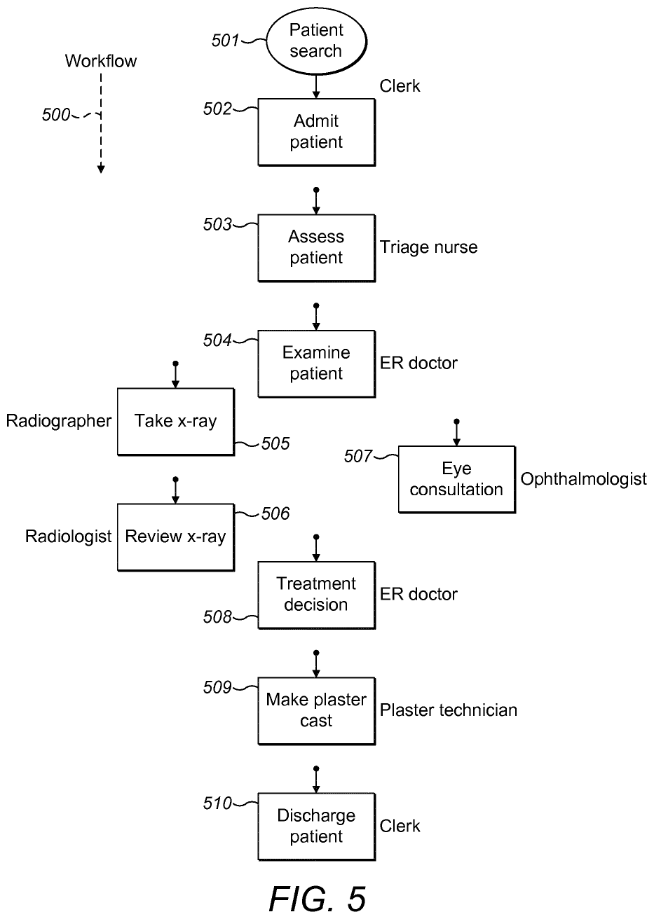

Through the sequential selection of actions by users of the system in the manner described herein, workflows may be performed in which each step is represented by one or more actions. For instance, in the hospital example described below with reference to FIG. 5, an action could be making an appointment for a patient through the computer system, performing a surgery on a patient (which may be reflected in the computer system through an action to update a data object instance representing the patient's record), or updating the address details of a patient. Each of these examples could be performed on a patient record data object instance.

A process definition may further comprise preconditions 802 which specify data (e.g. fields of a data object instance) which is to be used in the action from the data objects on which the action is configured to operate. For example, in the example of updating the address of a patient record, preconditions for that action may copy certain data from a patient record on which the action operates into the fields of a form which is to be completed by a user performing the address update action.

Workflows which are possible in the hospital example could be, for instance, handling a patient from admission to discharge, booking an appointment for a patient in an outpatient clinic, or performing an X-Ray. A workflow may itself comprise many smaller workflows: for example, in a workflow relating to the handling of a patient from admission to discharge at a hospital, an X-Ray may be required, the performance of which may itself represent a workflow.

The actions defined in the database represent steps in one or more workflows. However, unlike in conventional systems, the actions are not linked in the system so as to define a workflow: the link between actions is provided by the stakeholders (e.g. a user authenticated as a stakeholder of the system) who progress a workflow by selecting an action presented at a computer terminal. By ensuring that a stakeholder is defined who can perform each action representing a step of workflow and that those stakeholders are associated with the data object instances on which the actions are to be performed, a complete workflow can be achieved in an extremely flexible manner. At any point in a workflow, the next step is determined by a stakeholder of the system in the sense that any action available to that stakeholder can be performed on the data object instances with which they are associated. Through appropriate definition of the data with which a stakeholder is associated and the actions which can be performed by that stakeholder, a set of possible workflows are established at the system. This arrangement allows the system to model complex business processes in a business or organisation which involve skilled workers who may be able to take a range of possible decisions within a workflow. The framework provided by the computer system nonetheless ensures that only certain personnel can perform a given action and that such an action must be performed in accordance with the requirements of the process definition defining that action (e.g. that certain data is provided, that certain checks are performed).

A workflow comprises a sequence of actions. However, the database 203 does not define a workflow as a particular sequence of actions and the process definitions are not linked so as to define a predefined sequence of actions in the database. For example, the system may not include any data linking actions into a sequence, or otherwise indicating that one action is to be performed before or after another. The workflows enforced by the system emerge from the particular structure of system and the definition of the data objects and stakeholder objects as appropriate to the environment modelled by the system (e.g. a hospital), and in particular the roles in that environment which are represented by the stakeholders (e.g. the hospital staff).

The operation of the system in performing a workflow will now be illustrated by way of example with reference to FIGS. 4, 5 and the system shown in FIG. 2.

Consider the workflow 500 shown in FIG. 5 which represents a possible sequence of actions performed in managing a patient from admission to discharge in a hospital. In this example, database 203 includes stakeholder objects representing hospital staff who are involved in performing workflows which are to be managed by the system. The stakeholder objects define at least a clerk, triage nurse, ER doctor, radiographer, radiologist, consultant and a plaster technician. There may be one or more members of the hospital staff representing each of these stakeholders. Each member of staff may be represented by a particular instance of the appropriate stakeholder object. Each member of staff could be recognised by the system as being a particular stakeholder by means of the credentials with which they login to one of the terminals 206-208.

In this particular example, a person who has been injured in an accident is admitted into the emergency room by a clerk. The stakeholder object in database 203 defining a clerk allows the clerk to view all of the patient record data object instances available at the system in database 203. For instance, the clerk may by means of a desktop terminal search the patient records 501 by name in order to locate the record for the injured person (hereafter the patient). An example of the user interface 213 of the terminal by means of which the search is performed is shown in FIG. 4. The clerk enters search parameters (such as the injured person's name or other information fields of the patient record data objects) into the search fields 401 and, in response, the workflow manager 205 returns a list of the data object matches 402. Typically only some of the information held in each patient record would be displayed at the user interface—for example, just the name and date of birth of each matching patient.

The clerk can then select the patient record of the injured person (e.g. record 404 in FIG. 4). On this selection being performed, the workflow manager 205 processes the selected patient record data object in order to determine which (if any) actions can be performed on that data object instance by the clerk. Any actions which are available to the clerk are presented as actions 403 in the user interface. Were the clerk to select more than one patient record, the clerk might be presented with other actions, such as ‘Merge patient records’ to allow duplicate records to be merged, or ‘Link records’ for linking in the system members of a family. When more than one record is selected the workflow manger could be configured to display those actions which operate on multiple records (as could be specified in the data objects or the process definitions) and are common to the displayed data object instances. In other examples, the workflow manager could be configured to additionally or alternatively process the data object instances which are presented to the user in order to determine which (if any) actions can be performed on that data object instance, prior to or irrespective of the selection of any particular data object instance(s) by the user. This can be useful for workflows in which an action is performed on a number of data object instances at a time (e.g. all data object instances returned by a search or presented at the user interface).

In this example, the clerk is presented with an action ‘Admit patient’. By selecting this action 502 the clerk is presented with an admission form defined in the process definition held at the database for that action. The admission form might require the clerk to enter information such as a description of which part of the body is injured, their pain level, whether the patient is conscious, etc. In this case, the patient has an injured arm and eye. The admission form would be defined at the procedural data for the ‘Admit patient’ action. The procedural data could define, for example, the appearance of the form, its data fields, which fields are mandatory and which optional, the required format of the data for each field, routines for processing data entered into the form, etc. Depending on the nature of the action, any processing associated with the action and defined in the procedural data could be performed at the processor of the server and/or of the user terminal. Typically, processing relating to the presentation and entry of data could be performed at the terminal, and any substantive processing relating to data held at database 203 could be performed at the server. Procedural data held at a process definition could include routines or code for execution at the terminal and/or server.

The process definition may further include a set of preconditions which define data required by the respective action. The preconditions specify to the workflow manager which data is to be used from the selected data object(s). For example, when the clerk selects the ‘Admit patient’ action, the workflow manager automatically copies data fields specified in the preconditions of that action from the selected patient data record into an admission record data object instance. In this example, the fields might include the name, health system number, date of birth and address of the patient, with the workflow manager populating the form with that data according to the preconditions of the action. The ‘Admit patient’ action generates a new instance of an admission record data object linked to the patient record data object instance. Once the action is complete and the admission record has been generated, the clerk's role in admitting the patient is complete.

Conventional workflow management systems do not allow a user to search or browse their data and, on the basis of the data viewed or selected, present to the user actions which can be run on that data by the user. In conventional systems, actions can only be run by selecting a case which has been pushed to a user in accordance with a strict workflow which is predefined at the system. The user would then start a predetermined action on that case, again according to the predefined workflow.

A second stakeholder in the system is a triage nurse. The triage nurse stakeholder is associated with admission records. When a triage nurse user searches or views admission records at a user interface 213 of a terminal, the triage nurse is provided with the action ‘Assess patient’ on selecting a patient who has not yet been assessed. This is achieved because the admission record data object processed by the workflow manager specifies that a triage nurse can perform the ‘Assess patient’ action on the data object only when the object information of the data object indicates that the patient has not yet been assessed. In other words, the action is presented only when a context specified in the selected data object is satisfied.

The triage nurse selects the admission record for the injured patient admitted by the clerk and runs the ‘Assess patient’ action 503. The triage nurse then assesses the patient, potentially aided by information presented at the terminal in accordance with the process, and determines the severity of the injuries. The triage nurse could also have access (through the stakeholder object with which the triage nurse as a system user is associated) to the patient record data objects. In this example, the injuries are not considered by the nurse to be life threatening so the nurse adds relevant notes to the admission record and selects a medium priority queue for treating the patient in the emergency room.

Note that the triage nurse does not perform the ‘Assess patient’ action on the admission record data object as a result of a predefined workflow specified in the database and performed by the workflow manager. The triage nurse performs the ‘Assess patient’ action on the admission record data object because the nurse views the instances accessible to him or her, either by browsing or searching, and selects the action made available to him or her by the workflow manager. Thus, the database and workflow manager ensure that each user can only perform the actions available to them in accordance with the respective process definitions for those actions and the context in which the user views their data. In this manner, the system constrains users to perform only permitted workflows without being required to define those workflows in advance at the database.

An ER doctor next examines the patient, for example when the patient's admission record reaches the top of the queue displayed at the user interface of the doctor's terminal. ER doctors as a stakeholder are associated with admission record and patient record data objects. On selecting the admission record for the patient, the ER doctor is provided with the action ‘Examine patient’, which the doctor selects 504. This provides a form into which the doctor can make notes and also permits the doctor to view the patient's medical record (held in the object information of the patient record for the injured person) and the notes made by the triage nurse. Being a skilled worker, the ER doctor is not however led through a process defined for the ‘Examine patient’ action. The action represents the decision point at which the doctor freely determines the investigations to be performed on the patient and provides the doctor with the ability to order a range of investigations through the workflow management system (e.g. the procedural data for the action might provide a search interface at the terminal through which the doctor can request investigations, tests, scans and other procedures, and enter notes into the patient's medical record).

The doctor decides that an X-Ray of the patient's arm is required and that an ophthalmologist should take a look at the patient's eye. Selecting the appropriate options provided by the ‘Examine patient’ action causes the object information of the admission record to be modified to include the endorsements ‘X-ray required’ and ‘Ophthalmologist required’. The radiographer stakeholder could be configured to be provided with the action ‘Take X-Ray’ for admission records so endorsed which are viewed or selected by a radiographer user. The ophthalmologist stakeholder could be configured to be provided with the action ‘Eye consultation’ for admission records so endorsed which are viewed or selected by a radiographer user.

The user interface 213 of a terminal could be configured in dependence on the stakeholder with which the user of the terminal is associated. For example, on a radiographer logging into a terminal with their user credentials, the terminal could be configured to display a list of admission records which include the endorsement ‘X-ray required’. To give a second example, on an ophthalmologist logging into a terminal with their user credentials, the terminal could be configured to display a list of admission records which include the endorsement ‘Ophthalmologist required’. This could be achieved through the use of user profiles held at the system (e.g. at the database 203) for the users of the system which specify the stakeholder objects with which each user is associated. By requiring a user of the system to authenticate themselves as a particular user (e.g. by logging in with their credentials), the system could load the appropriate user profile and hence the user interface associated with the associated stakeholder(s). The particular configuration of the user interface for a stakeholder could be stored in interface configuration data at the respective stakeholder object.

The radiographer selects the ‘Take X-Ray’ action 505 in respect of the patient's admission record which causes an X-Ray data object to be generated on which the ‘Take X-Ray’ action is performed. The X-Ray data object is linked to the patient's admission record. The resulting X-Ray image is stored in the X-Ray data object. A radiologist stakeholder is associated with all X-Ray data objects and hence can view the X-Ray data object linked to the patient's admission record. On selecting the X-Ray object, the radiologist is presented by the workflow manager operating on the X-Ray data object the action ‘Review X-Ray’. The radiologist selects this action 506 and enters their finding that the patient's arm is broken into a form presented by their terminal in accordance with the definition of the action in its respective process definition.

The ophthalmologist could similarly be able to run the ‘Eye consultation’ action 507 when selecting the patient's admission record. The ophthalmologist enters their finding that the eye has suffered only minor damage which needs no specific treatment into a form presented by the action and the notes are then stored in the admission record.

As each investigation is completed, the respective action could endorse the admission record so as to permit the ER doctor to determine when the investigations have completed. At that point, the ER doctor progresses the workflow by selecting the ‘Treatment decision’ action 508 on the patient's admission record. The doctor could determine when investigations have been completed by searching at their terminal for those records whose investigations are marked as completed by means of an appropriate endorsement (e.g. a flag or any other kind of indicator). The doctor can view the notes of the ophthalmologist and the X-Ray image linked to the admission record in the X-Ray data object. The doctor determines that the broken arm should be put into a plaster cast and so orders a forearm plaster cast from a list of treatments made available by the ‘Treatment decision’ action. This causes the admission record to be modified to show that a plaster cast is required.

A plaster technician uses a terminal which is configured to present all admission records which have been endorsed to show that a plaster cast is required. On the patient reaching the top of their work queue provided by the user interface at their terminal, the plaster technician selects that patient and is provided with the option to run the action ‘Make plaster cast’ 509. This action presents the plaster technician with the notes made by the doctor in the admission record identifying the type of cast required. The plaster technician makes the cast and marks the job complete in the action.

Finally the clerk can discharge the patient once all of the treatments requested by the doctor have been completed. This is achieved by configuring the admission record data object to cause the workflow manager to present to a clerk the action ‘Discharge patient’ 510 only when the clerk selects the admission record of a patient for which all of the requested treatments are marked as completed.

Such a complex workflow cannot be achieved in conventional systems because the number of possible investigations and treatments is enormous and it would not be realistic to specify strict processes for every possible combination of investigations and treatments that a patient might require. Furthermore, because the computer system makes actions available to a user (a) according to the capabilities defined in the system for stakeholders to which the user belongs and (b) the context represented by the data to which that stakeholder has access, the system provides an architecture which ensures that workflows are appropriately performed.

For example, in order to enable a particular workflow to be performed, the stakeholder and data objects may be configured such that for each step of the workflow there is defined at least one stakeholder associated with the data objects on which that step is to be performed who is permitted to select the action representing that step. In order to prevent stakeholders from performing certain actions on their data, their data objects may be configured so as to not specify that the stakeholder can perform those actions. In order to prevent a stakeholder from accessing certain data objects or instances of a data object, the stakeholder object or instance of a stakeholder object representing that stakeholder may be configured so as to not specify those data objects as objects which the stakeholder can access.

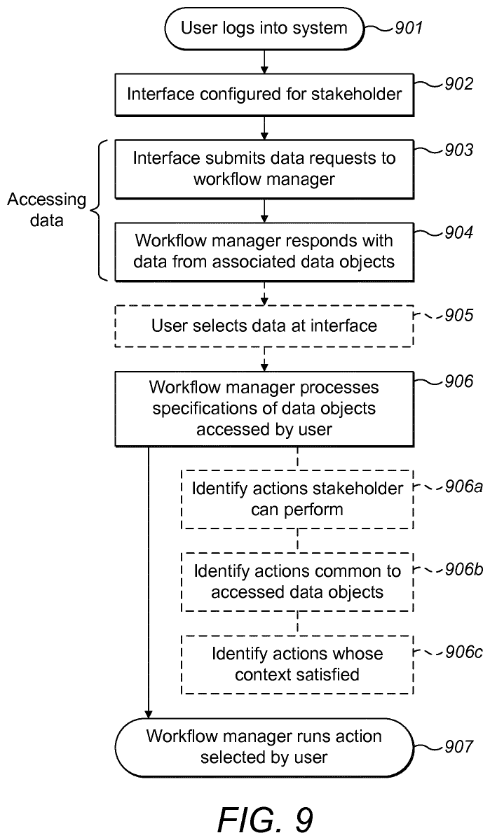

An example of the operation of the system shown in FIG. 2 when a user performs an action within a workflow will now be described with reference to FIG. 9. At 901, the user logs into the system at a user terminal, e.g. by entering their login credentials into a user interface 213 provided as a web application. The identity of the user could be verified at the user terminal 206 or the server 201. In this example, the workflow manager has access to a stored profile for each user which indicates to the system the stakeholder that the user represents. When the user logs into the system, the workflow therefore knows which stakeholder(s) the user represents and hence which stakeholder object(s) identify the data to which the user is to be permitted to access.

In response to the user logging into the system, at 902 the workflow manager running at processor 202 provides to the user terminal data stored at the user's profile or the associated stakeholder object which defines how the user interface 213 at the terminal should configured itself for that user (e.g. the appearance of the user interface, what search options should be presented, and whether the interface should attempt to load by default any data from the data object instances associated with the stakeholder represented by the user).

At 903, the user submits a data request to the workflow manager by means of the user interface. For example: the user could browse their data, with the user interface being configured to request information from the workflow manager which satisfies the browsing performed by the user; or, the user could perform a search, with the search request entered into the user interface being passed to the workflow manager which performs the search within the data objects associated with the stakeholder represented by the user. The workflow manager satisfies the data requests received from the user interface (904) by returning the requested data from the data objects of the stakeholder represented by the user according to the associated data objects defined at the respective stakeholder object (e.g. 602 in stakeholder object 600 of FIG. 6). The user interface presents the returned data at the user interface. The workflow manager could be configured to return data restricted to the data objects of the stakeholder according to any suitable technique, such as by means of a suitable query into database 203 which holds the data objects (e.g. the query could include a list of the data objects/instances which are identified by the stakeholder object).

Browsing data could include the user interface loading data by default on the user logging on to a terminal (e.g. in accordance with a data stored at the user's profile or at the associated stakeholder objects).

Steps 903 and 904 in the flowchart example represent a stakeholder (represented by the user) accessing their associated data objects. A stakeholder could alternatively or additionally access their data objects by selecting data presented to them (e.g. as the result of browsing or a search). This is indicated by flowchart step 905.

When a user accesses data at the user interface, whether through browsing, searching or selecting data presented at the interface, the user interface indicates to the workflow manager what data is being accessed. When browsing or searching, this information is naturally provided to the workflow manager in order to cause the workflow manager to return the appropriate data. When the user selects data presented to them at the user interface, the interface indicates to the workflow manager the data which is selected.

At 905, the workflow manager processes the specifications of the data objects and/or instances at which the accessed data is held (e.g. 702 in the data object 700 shown in FIG. 7) in order to identify the set of actions which can be performed on those data objects and which the stakeholder represented by the user is permitted to perform. Such processing could be performed in any suitable manner. For example, the workflow manager could firstly filter the actions listed at the accessed data objects to identify a first set of actions which the stakeholder is permitted to perform (906a); the workflow manager could then filter the first set of actions to identify those actions which are common to all of the accessed data objects (906b) and hence generate a second set of actions (this step may not be required if only one data object is selected). The accessed data objects may further specify a context (e.g. a set of criteria as shown in 702) which must be satisfied in order for the action to be presented to the user. The workflow manager would therefore finally filter the second set of actions in order to identify a final set of actions (906c) whose context is satisfied by predefined criteria of the data objects (typically although not necessarily limited to the accessed data objects) for presentation to the user at the user interface in response to the accessing of data by the user. For instance, in the hospital example given above the patient data objects could additionally specify a ‘Register patient death’ action which any doctor (the stakeholder) can perform but which is only available to the doctor when selecting a patient record with has the criterion ‘IsDead’ set to true.

At 906, the user interface presents the final set of actions determined by the workflow manager to the user. Steps 903-906 can be repeated as the data accessed by the user changes so as to ensure that the user is always presented with an appropriate set of actions according to the data browsed, searched, selected or otherwise accessed by the user. At 907, the user selects an action presented at the user interface which causes the workflow manager to access the process definition stored for the action at the database and run the action according to the procedural data defining the action. This procedural data could, for example, define one or more of: a form the user is to complete; a set of checks to be performed against data entered by the user; a process to generate a new instance of a data object; a process to modify a data object. At least some of the procedural data could be run at the user terminal (e.g. this is where a form would be presented to the user for completion). The workflow manager could be configured to initiate an action with data from data objects held at the database according to preconditions present at the process definition for the action.

In this manner the users of the system are empowered to follow appropriate workflows without being overly constrained by predefined sequences of actions.

Designing Workflows

In order to enable workflow manager 205 to enforce workflows appropriate to a business scenario, it is necessary to define in database 203 suitable actions for each stakeholder and, optionally, rules for presenting those actions to the stakeholder. Such definitions may be made by at design time prior to the performance of workflows by the workflow manager.

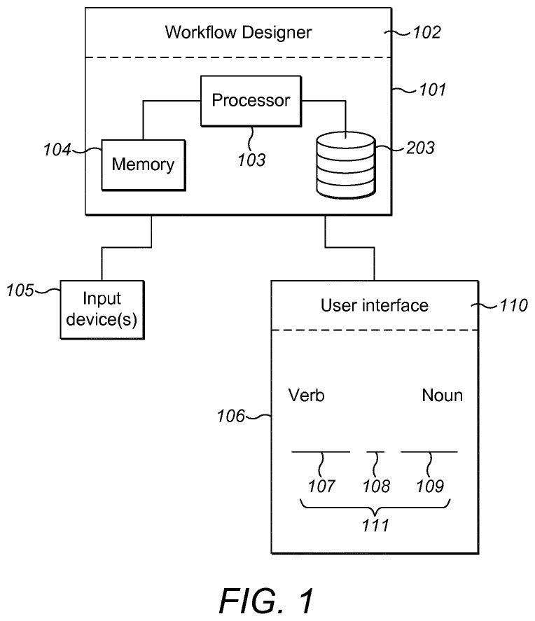

A computer system may be provided to enable a designer to generate the necessary data structures at database 203. A suitable computer system 101 is shown in FIG. 1 at which workflow designer software 102 runs. The computer system comprises a processor 103 and memory 104 for supporting the workflow designer software. A user interface 110 of the software 102 may be provided at a display screen 106 so as to enable a designer to interact with the computer system. The user interface may be generated by processor 103 according to the software code representing workflow designer. The designer may provide input to the computer system by means of input device 105, which could be, for example, one or more of a keyboard, mouse, and a touchscreen interface of display 106. In one embodiment, the computer system 101, display screen 106 and input device(s) 105 represent a workstation configured for designing workflows for use at workflow manager 205.

Workflow database 203 at which appropriate data structures for the is accessible to the workflow designer 102. In FIG. 1 the database 203 is schematically shown and may be provided in any location accessible to the computer system 101 (e.g. at a data store accessible over a network or the internet, at a data store local to the computer system, etc.).

The database 203 may have the structure illustrated in FIG. 3 and described above, and may comprise one or more of the data structures shown in FIGS. 6 to 8 and described above.

The workflow designer shown in FIG. 1 enables a designer (a user of the workflow designer software) to define stakeholder objects, data objects and actions appropriate to the real-world business processes which the workflows are to represent. As is shown in FIG. 6-8, in order to be more than merely empty data structures, the properties of the stakeholder objects, data objects and actions must be carefully defined so as to link stakeholders to their data objects (or instances thereof) and actions in the manner described above. One way of defining the properties of these data structures is to provide a design interface which enables a designer to set the properties of data structures by entering data according to some predefined syntax—for example, expressions which can be interpreted by workflow manager 205.

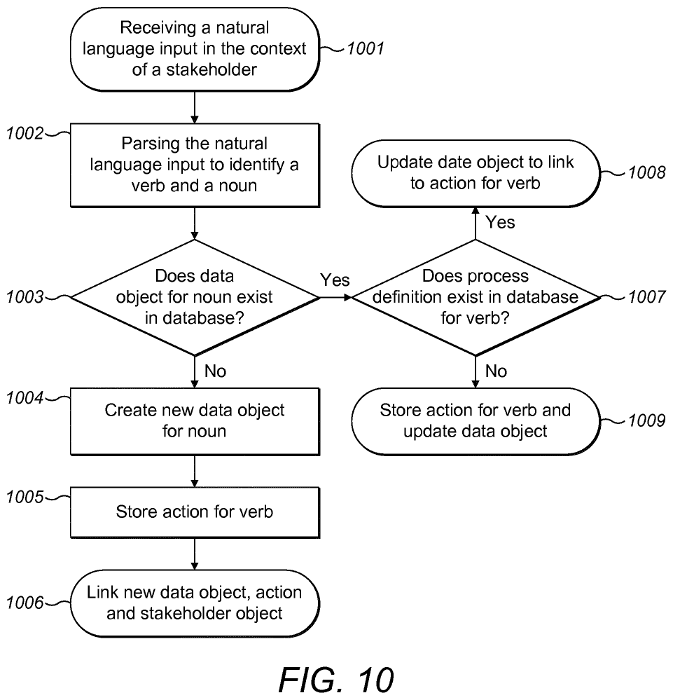

However, such an approach requires the designer to both understand the technical requirements of the syntax and to carefully plan out in advance the workflows which are to be enforced by the workflow manager. This can present difficulties for non-technical personnel who are not experienced writing technical code according to a strict syntax. An improved approach will now be described with reference to an exemplary workflow shown in FIG. 10.

The user interface 110 shown in FIG. 1 is configured to allow the designer to enter a natural language phrase 111 expressing an action which may be performed on a particular type of data object (step 1001 in FIG. 10). The natural language phrase includes at least a verb 107, indicating what is to be done, and a noun 109, which indicates the type of entity on which the action is to be performed. For example, in the context of the hospital example above, the designer may enter (using the input device(s) 105) “Book an X-Ray”. A “connector” element 108 may be present in the phrase between the verb and noun which may be, for example, an article or preposition in the English language. The connector may be ignored by the workflow designer but it can be helpful to allow the designer to enter such a connector since this allows the designer to enter a natural phrase whose meaning is clear to them. It can be helpful to indicate to the designer in the user interface that the natural language phrase entered should be in the form verb-connector-noun (e.g. through appropriate labelling of fields 107-109, or by providing instructions to the designer).

The workflow designer parses the natural language phrase so as to identify the verb and noun in the phrase (1002 in FIG. 10). In some examples, this may be achieved by configuring the user interface to provide separate fields for the verb 107 and noun 109. This minimises the analysis of the phrase required of by workflow designer 102 in order to determine which words of the phrase 111 are the verb and noun. Further, the use of such fields can be helpful in guiding the designer into entering a suitable phrase of the form verb-connector-noun. In other examples, the user interface may provide a single field into which the phrase is entered by the designer, with the workflow designer being configured to determine which word(s) of the phrase represent the verb and which word(s) represent the noun. Such processing may determine the nature of the words from one or more of their position in the phrase, through the use of dictionaries of verbs and/or nouns, and by identifying the connector and, assuming the connector separates the verb and noun, using that knowledge to identify the verb and noun parts of the phrase. Suitable algorithms for performing such processing are known from the field of natural language processing.

The workflow designer is configured to check whether a data object exists in workflow database 203 for the noun (1003 in FIG. 10). This can be achieved by checking for a data object which is associated with the noun—for example, by looking for a data object which has the noun as its name, title or other identifier, or which is tagged with that noun. It can be useful to arrange that data objects can be provided with multiple nouns as tags so as to enable a data object to be associated with more than one noun. This can be helpful where multiple nouns may refer to the same entity—e.g. a doctor may also be referred to as a medic; a CAT scan may also be referred to as a CT scan.

Any suitable searching algorithm may be used by the workflow designer to search for data objects associated with the noun in the workflow database.

If a data object associated with the noun is not already present in the workflow database, the workflow designer is configured to automatically create a data object associated with the noun in the workflow database 203 (1004 in FIG. 10). The data object would preferably take the name of the noun or a name derived from the noun. The data object may be tagged with the noun so as to associate the noun with the data object.

The new data object may be stored based on a predefined generic data object—for example, a default data object having predefined characteristics may be available to the workflow designer (e.g. stored in working memory 104 or at another data store). Alternatively the user may be able to specify one of a set of generic data objects, depending on what the data object is to represent—e.g. according to whether it is to hold image data or appointment details, or which department the data object relates to etc. For instance, in the hospital example, the user could choose from default data object types such as: appointment, test results (e.g. X-Ray or blood test), paediatric, orthopaedic, geriatric medicine etc. Different types of data objects could include different default sets of fields for dates, image data, patient record information etc.

A new action is also created for the verb entered by the designer (1005 in FIG. 10). The new action may be created by storing a new process definition 800 at workflow database 203, in accordance with FIG. 8 discussed above. The new action is linked to the new data object such that the action is permitted to run on the data object (1006 in FIG. 10). For example, the specification data 702 of the data object may be defined so as to permit the newly created action to operate on the instances of that data object.

The new action would preferably take the name of the verb or a name derived from the verb. The action may be tagged with the verb so as to associate the verb with the action. The new action may be stored based on a predefined process definition—for example, a default process definition having predefined characteristics may be available to the workflow designer (e.g. stored in working memory 104 or at another data store). Alternatively the user may be able to specify one of a set of generic process definitions, depending on what the action is to perform. For instance, the action may be for performing a process (e.g. a fixed step in a workflow), a form (e.g. a window intended to receive input from the user at runtime), or a rule (e.g. code which will be automatically executed on workflow data). A different generic process definition may be provided for each type of action and used to generate the new action on the natural language phrase being entered.

Prior to or when entering the natural language phrase, the designer further specifies the stakeholder(s) who can perform the action. These stakeholder(s) are identified in the specification data 702 of the data object that is created so as to ensure that only the appropriate stakeholders can perform the workflow step expressed by the natural language phrase.

Prior to or when entering the natural language phrase, the designer may further specify a context which determines when the action is made available to the stakeholder at runtime. The context may comprise one or more conditions which are verified at runtime; only if the condition(s) are satisfied is the action made available to the stakeholder through the user interface of the workflow manager 205. For example, when developing workflows for an obstetrics department of a hospital, a designer may add a workflow step to schedule a pregnancy checkup for a patient by entering the natural language phrase “Schedule a pregnancy checkup” (where “schedule” is the verb and “pregnancy checkup” is the noun). In order for this to make sense, the patient stakeholder must be pregnant—the context condition may therefore comprise a check to determine whether the stakeholder definition of the stakeholder instance representing the patient at runtime indicates that the patient is pregnant (e.g. whether a IsPregnant flag is set to “true”).

If a data object associated with the noun already exists in the workflow database, the workflow designer is configured to check whether a process definition exists for an action which is associated with the entered verb (1007 in FIG. 10). If such a process definition exists then the process definition and data object are linked by updating the data object (e.g. field 702) to allow the action corresponding to the process definition to be performed on the data object (1008 in FIG. 10). If such a process definition does not exist, a new process definition is created in the workflow database in the manner described above and linked to the existing data object so as to permit the newly created action to operate on the instances of that data object (1009 in FIG. 10).

It is advantageous if a stakeholder permitted to perform a workflow step is implicit from how the natural language phrase is entered into the user interface. For example, the workflow designer may be configured to allow a designer to enter a natural language phrase on selecting one or more stakeholder objects, or from a user interface which represents a stakeholder object. This assist a designer in developing appropriate workflows because the designer will often consider a workflow from the point of who in an organisation will be performing each of the steps.

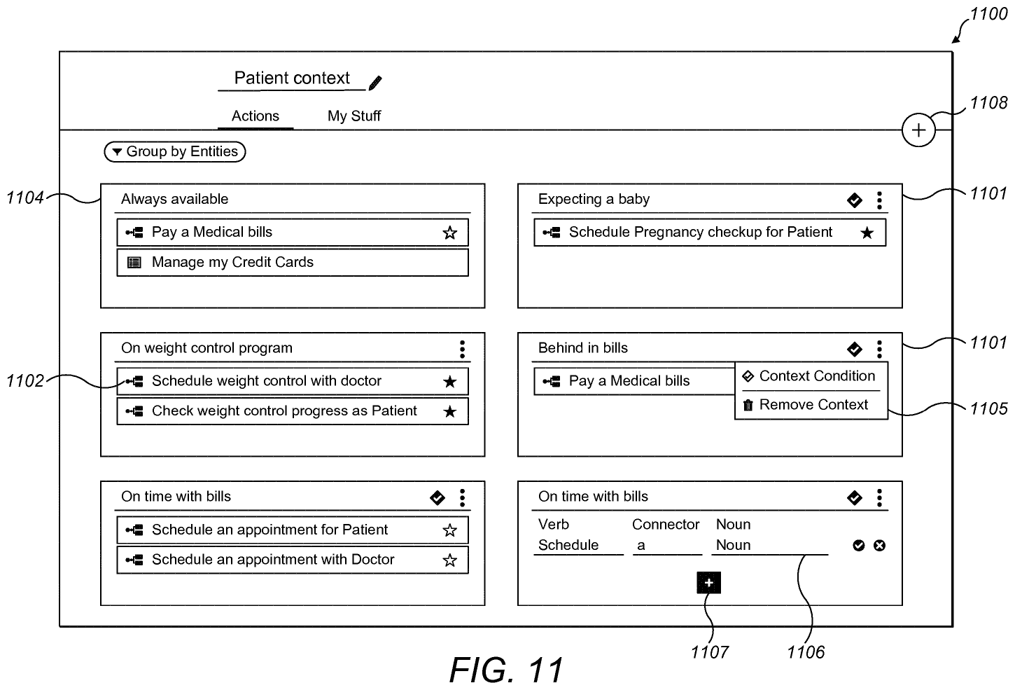

It is particularly advantageous if the workflow designer is configured to provide a context user interface for each stakeholder. A context user interface groups together actions which may be performed by the stakeholder according to a context condition which causes those actions to be made available to the user at runtime. This approach is illustrated in FIG. 11.

FIG. 11 shows a context screen 1100 for a patient stakeholder. The screen includes groups 1101 of workflow steps 1102. Each group has an associated context condition 1105. The workflow designer may be configured to automatically create each group when a new workflow step is created which does not belong to an existing group—such a new group may be created in dependence on the context condition entered for the workflow step. The designer may be prompted at user interface 110 to enter a suitable title for the group based on the context condition. New steps which share a context condition with an existing group may be added to that group. A group 1104 may be provided for any workflow steps which are always available to the stakeholder—these need not be provided with a context condition. Each group may be a convenience provided at design time and need not be present in the structure of workflow database 203 on which the workflow manager operates at runtime.

The context screen 1100 may provide a user interface by which a designer can enter a natural language phrase indicating to the system a new workflow step the designer would like to create. Such a user interface may be made available to the designer for an existing group 1101 (e.g. by clicking button 1107) or on creating a new group (e.g. by clicking button 1108 and entering a context condition). The designer may then be presented with one or more fields 1106 in which to enter a natural language phrase and potentially cause a new data object and action to be generated in the workflow database. The one or more fields 1106 are adapted for receiving a natural language phrase 111 as described above with respect to FIG. 1. The fields may comprise verb, connector and noun fields 107-109 as shown in FIGS. 1 and 11.

The approach presented in FIG. 11 and described above is most advantageous since both the stakeholder who may perform the workflow step and the context condition which is to determine when the workflow step is presented to that stakeholder at runtime are implicit from the location at which the designer enters the natural language phrase. This approach makes it straightforward for a designer with little technical understanding to generate the data objects and process definitions required by a workflow manager in order to enforce the desired workflow.

Once the new data object and process definition have been created in the workflow database in response to the entry of a natural language phrase, the designer may further edit the parameters of the data object and process definition using the conventional user interface for configured those elements of a workflow database. This is a more straightforward task once the required data object and process definition are present in the system and linked to the appropriate stakeholder and context condition. For instance, it may be necessary for the designer to enter procedural data for a process definition, as well as further information defining the particular characteristics of the newly created data object and action (initially the data object and process definition would have the generic characteristics of the default data object or process definition on which they are based). Such steps could be performed by a technical designer once the general workflow steps have been put in place according to the principles described herein.

In the examples described herein, the data objects, stakeholder objects and actions are held in a database, but more generally such data could be provided anywhere accessible to the system and in any suitable form. The term object is to be understood as to not limit the database to being an object database; the term object is used broadly to refer to any collection of data having the information content described herein.

The computer systems of FIGS. 1 and 2 are shown as comprising a number of functional blocks. This is schematic only and is not intended to define a strict division between different logic elements of such entities. Each functional block may be provided in any suitable manner.

Generally, any of the functions, methods, techniques or components described above can be implemented in software, firmware, hardware (e.g., fixed logic circuitry), or any combination thereof. The terms “module,” “functionality,” “component”, “element”, “unit”, “block” and “logic” may be used herein to generally represent software, firmware, hardware, or any combination thereof. In the case of a software implementation, the module, functionality, component, element, unit, block or logic represents program code that performs the specified tasks when executed on a processor. The algorithms and methods described herein could be performed by one or more processors executing code that causes the processor(s) to perform the algorithms/methods. Examples of a computer-readable storage medium include a random-access memory (RAM), read-only memory (ROM), an optical disc, flash memory, hard disk memory, and other memory devices that may use magnetic, optical, and other techniques to store instructions or other data and that can be accessed by a machine.

The terms computer program code and computer readable instructions as used herein refer to any kind of executable code for processors, including code expressed in a machine language, an interpreted language or a scripting language. Executable code includes binary code, machine code, bytecode, and code expressed in a programming language code such as C, Java or OpenCL. Executable code may be, for example, any kind of software, firmware, script, module or library which, when suitably executed, processed, interpreted, compiled, executed at a virtual machine or other software environment, cause a processor of the computer system at which the executable code is supported to perform the tasks specified by the code.

A processor, computer, or computer system may be any kind of device, machine or dedicated circuit, or collection or portion thereof, with processing capability such that it can execute instructions. A processor may be any kind of general purpose or dedicated processor, such as a CPU, GPU, System-on-chip, state machine, media processor, an application-specific integrated circuit (ASIC), a programmable logic array, a field-programmable gate array (FPGA), or the like. A computer or computer system may comprise one or more processors.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein. In view of the foregoing description it will be evident to a person skilled in the art that various modifications may be made within the scope of the invention.

1. A computer-implemented method for designing a workflow for use in workflow management software having access to a workflow database that includes one or more data objects, actions for operation on data objects, and a plurality of stakeholder objects each associated with (a) one or more of the data objects and (b) one or more of the actions, the method comprising:
receiving at a user interface of a computer system a natural language input and an indication of a stakeholder represented in the workflow database by a stakeholder object;
parsing the natural language input at a processor so as to identify a verb and a noun in the natural language input;
verifying whether a data object corresponding to the noun exists in the workflow database; and
if such an entity type does not exist in the workflow database:
using a predefined data object, creating in the workflow database a new data object for the identified noun;
using a predefined process definition, storing in the workflow database an action for the identified verb; and
linking in the workflow database the new data object, the action for the identified verb, and the stakeholder object such that, when the workflow management software is in use by the stakeholder, the stakeholder is permitted to operate the action on an instance of the new data object associated with the stakeholder.

2. A computer-implemented method as claimed in claim 1, wherein each stakeholder is permitted to operate on a data object associated with the stakeholder only those actions which are linked to that data object and the stakeholder object representing the stakeholder.

3. A computer-implemented method as claimed in claim 1 or 2, wherein the parsing the natural language input at a processor is performed in dependence on one or more of: the position of the words in the natural language input; the presence of a connector word delineating a verb and noun; in which fields of a user interface the words of the natural language input are present.

4. A computer-implemented method as claimed in any preceding claim, wherein the parsing the natural language input at a processor comprises processing the natural language input using a natural language processing algorithm configured to identify a verb and a noun on which the verb syntactically operates.

5. A computer-implemented method as claimed in any preceding claim, wherein the verifying whether a data object corresponding to the noun exists in the workflow database comprises checking the data objects defined in the workflow database for a data object satisfying one or more of the following the conditions: being identified by the noun; tagged with the noun; having an identifier predefined as being equivalent to the noun.

6. A computer-implemented method as claimed in any preceding claim, wherein the linking in the workflow database comprises defining in the new data object that the stakeholder represented by the stakeholder object is permitted to operate the identified verb action on the new data object.

7. A computer-implemented method as claimed in any preceding claim, wherein the receiving at a user interface further comprises receiving an indication of a context condition and the linking in the workflow database comprises defining in the new data object that the stakeholder represented by the stakeholder object is permitted to operate the identified verb action on the new data object only when the context condition is satisfied.

8. A computer-implemented method as claimed in any preceding claim, wherein the natural language input is received at a user interface relating to a stakeholder, the receiving an indication of a stakeholder comprising identifying the stakeholder as the stakeholder to which the user interface relates.

9. A computer-implemented method as claimed in any preceding claim, wherein the user interface relating to a stakeholder is a design interface configured to allow a user to modify the stakeholder object representing the stakeholder.

10. A computer-implemented method as claimed in any preceding claim, wherein the predefined data object is a default data object, the new data object inheriting the properties of the default data object and being identified amongst the data objects of the workflow database by the noun.

11. A computer-implemented method as claimed in any preceding claim, wherein the predefined process definition is a default process definition, the action inheriting the properties of the default process definition and being identified amongst the actions of the workflow database by the verb.

12. A computer-implemented method as claimed in any preceding claim, wherein the method comprises, subsequent to the linking, prompting the user to edit attributes of one or more of the new data object and the action for the identified verb.

13. A computer-implemented method as claimed in any preceding claim, wherein the process definitions are defined at a process layer of the workflow database and the data objects are defined at a data layer of the workflow database.

14. A computer system for designing a workflow for use in workflow management software having access to a workflow database that includes one or more data objects, actions for operation on data objects, and a plurality of stakeholder objects each associated with (a) one or more of the data objects and (b) one or more of the actions, the system comprising:
a data store comprising the workflow database;
a user interface for receiving a natural language input and an indication of a stakeholder represented in the workflow database by a stakeholder object;
a processor configured to:
parse the natural language input so as to identify a verb and a noun in the natural language input;
verify whether a data object corresponding to the noun exists in the workflow database; and
if such an entity type does not exist in the workflow database:
using a predefined data object, creating in the workflow database a new data object for the identified noun;
using a predefined process definition, storing in the workflow database an action for the identified verb; and
linking in the workflow database the new data object, the action for the identified verb, and the stakeholder object such that, when the workflow management software is in use by the stakeholder, the stakeholder is permitted to operate the action on an instance of the new data object associated with the stakeholder.

15. Computer program code for performing a method as claimed in any of claims 1 to 14.

16. A non-transitory computer readable storage medium having stored thereon computer readable instructions that, when executed at a computer system, cause the computer system to perform the method as claimed in any of claims 1 to 14.

17. A computer system substantially as described herein with reference to any of FIGS. 1 to 11.
